(19) United States
(12) Patent Application Publication
(10) Pub. No.: US 2021/0405071 A1
(43) Pub. Date: Dec. 30, 2021

(54) BIOMARKERS FOR PREDICTING IMMINENT OVULATION OR REPRODUCTIVE COMPETENCE IN MARES

(71) Applicant: MOTE MARINE LABORATORY, INC., Sarasota, FL (US)

(72) Inventor: Dana L. Wetzel, Sarasota, FL (US)

(73) Assignee: MOTE MARINE LABORATORY, INC., Sarasota, FL (US)

(21) Appl. No.: 17/290,030

(22) PCT Filed: Oct. 29, 2019
(86) PCT No.: PCT/US2019/058495

§ 371 Date: Apr. 29, 2021

Related U.S. Application Data
(60) Provisional application No. 62/752,056, filed on Oct. 29, 2018.

(51) Int. Cl. G01N 33/74; A61D 17/00

(52) U.S. Cl. CPC G01N 33/74; G01N 2333/51; G01N 2333/495; A61D 17/002

(57) ABSTRACT

Broodmare imminence of ovulation and reproductive competence may be determined by collecting blood, serum or plasma samples from a mare over two or more days in such mare's estrous cycle; obtaining a set of concentration measurements of one or more peptide hormones of the TGF-β superfamily in such samples over such two or more days; detecting an onset of a change in the relative magnitude or slope of successive concentration measurements of such one or more peptide hormones during such cycle; and coordinating live cover or artificial insemination of such mare to occur no later than three days after such onset. The disclosed method may be performed using an associated system that measures, processes and stores the measurements. The system may detect and optionally provide notification of the onset of such change.

### TECHNICAL FIELD

This invention relates to horse breeding.

### BACKGROUND

Although a number of animal species are selectively bred, equine breeding involves special considerations. Successful equine breeding is most likely to occur if natural copulation (generally referred to as “live cover”) or artificial insemination occurs during a short time window running from approximately two days before ovulation until the day of ovulation. Outside this time window the success rate typically decreases dramatically or disappears. Even within this window, successful pregnancy rates vary significantly from day to day, with the highest success rates arising from live cover or artificial insemination about two days before ovulation and the lowest success rates arising from live cover or artificial insemination on the day of ovulation.

There have been attempts to measure estrogen levels, progesterone levels or other hormones in order to detect or predict the onset of equine ovulation, as discussed for example in U.S. Pat. No. 5,460,976 (O'Connor) and U.S. Pat. No. 5,693,534 (Alak et al.), U.S. Patent Application Publication No. US 2009/0305947 A1 (Iscovich) and European Patent Application No. EP 2465469 A1 (Vankrieken et al.). However, these approaches do not appear to have entered into widespread use. Instead, veterinarians are generally called upon to determine if equine ovulation has occurred or to estimate when it mi t occur. Generally this is done via ultrasound analysis (ultrasonography), palpation (gloved manual examination of the broodmare's ovaries, accessed via the rectal canal) or by visual examination of the cervix using a vaginal speculum. These techniques can require appreciable time, skill and expense. Advance scheduling of the veterinarian and other personnel is usually required, and repeated visits may sometimes be needed for reassessment over the course of several days. Hormonal injections may also or instead be needed to promote ovulation within a specific timeframe.

Once it has been determined that a mare is ready for breeding, it is also necessary to make arrangements for live cover by the sire or for artificial insemination using a suitable semen sample. As is the ease with detection or prediction of ovulation, making arrangements for breeding can also be time critical. For example, in the U.S., live cover breeding is required in order for thoroughbred offspring to likewise be designated as thoroughbreds. Arranging live cover usually requires transporting the mare (and sometimes one or more of her foals as well) to a breeding shed via trailer or truck, arranging participation by the sire (and if need be his transportation via trailer or truck), scheduling time at the breeding shed and arranging participation by specialized horse-handling personnel who may be needed to prevent injury to the sire or mare.

If artificial insemination is employed (as permitted for example, for standardbred horses, quarter horses, Arabians and other non-thoroughbred breeds), the desired semen samples must be ordered and kept in a viable state until they can be used. Fresh horse semen typically can be kept under refrigeration for no more than about 48 hours, and frozen semen must be allowed time to thaw and then used promptly. The life span of frozen-thawed semen may be even shorter and requires insemination very close to ovulation (e.g., from 12-24 hours before ovulation to about 6 hours after ovulation) for a reasonable chance of success. This further increases costs as repeated and frequent veterinary examinations are necessary to target this narrow window. In practice, the window is often treated as closed at ovulation, as it is difficult to determine by veterinary exam whether the mare is still within an acceptable time frame for fertility.

The financial stakes are considerable. For example, in Kentucky, approximately 22,000 thoroughbred mares are boarded on local farms in the three countries surrounding Lexington, Ky. so they can be bred via live cover to one of approximately 600 locally-boarded thoroughbred stallions. Stud fees can range as high as $300,000 per live foal, and revenue related to horse breeding for the state of Kentucky as a whole may be as much as $3 billion per year.

If live cover or artificial insemination is unsuccessful, the above-described steps will need to be repeated. Doing so involves additional time and expense, and possible further risks of injury to the sire or mare. The associated costs of all these measures and steps can add up quickly, and the more times a mare is brought to live cover or inseminated to achieve a pregnancy, the more money will be required. Not every mare becomes pregnant in every cycle. For example, the average conception rate via artificial insemination is about 60%, which may mean that two to three cycles will be needed to achieve pregnancy in 90% of mares. The associated veterinary costs can easily double or triple if the imminence of ovulation cannot be ascertained.

Some ovulating mares have low reproductive competence, that is, a less than typical ability to conceive and successfully complete pregnancy and delivery. This may be due to a variety of causes including infertility or low fertility, anatomical defects or deficiencies, and susceptibility to infection. Thus although such mares may be technically be said to be ready for breeding, the likelihood of conception or successful birth may be low and it consequently may be better to focus on breeding other mares from a herd it can be difficult to assess reproductive competence and in some cases it has been necessary to use trial and error methods. Doing so can require considerable time and expense even if contingent stud fees based on delivery of a live foal are not charged.

There consequently remains an unmet need for more effective and efficient methods and devices for assessing fertility potential and reproductive competence in mares.

### SUMMARY

The present invention provides, in one aspect, a method for assessing the imminence of mare ovulation and reproductive competence, which method comprises collecting blood, serum or plasma samples from a mare over two or more days in such mare's estrous cycle; obtaining a set of concentration measurements of one or more peptide hormones of the transforming growth factor-beta superfamily (TG-β superfamily) in such samples over such two or more days; detecting an onset of a change in the relative magnitude or slope of successive concentration measurements of such one or more peptide hormones during such cycle; and coordinating live cover or artificial insemination of such mare to occur no later than three days after such onset. The disclosed method has been used to correctly predict mare ovulation and reproductive competence for which other methods of analysis yielded inaccurate predictions.

The invention provides, in another aspect, a system for assessing the imminence of mare ovulation and reproductive competence, comprising a detector configured to measure concentrations of one or more peptide hormones of the TGF-β superfamily in a plurality of blood, serum or plasma samples obtained on two or more days during such mare's estrous cycle; a processor; storage for a set of measured concentrations and sampling dates of such peptide hormones in such samples; and an engine for detecting an onset of a change in the relative magnitude or slope of successive concentration measurements of such one or more peptide hormones during such cycle.

The above summary is not intended to describe each illustrated embodiment or every implementation of the subject matter hereof. The figures and the detailed description that follow more particularly exemplify various embodiments.

### BRIEF DESCRIPTION OF THE DRAWING

The disclosed subject matter may be more completely understood in consideration of the following detailed description of various embodiments in connection with the accompanying Drawing figures, in which:

FIG. 1 is a schematic view of a system for assessing the imminence of mare ovulation and reproductive competence;

FIG. 2 is a block diagram of a system for assessing the imminence of mare ovulation and reproductive competence;

FIG. 3a through FIG. 3c are graphs of Inhibin A concentrations in plasma from three Arabian mares over an estrous cycle time period;

FIG. 4 is a graph of Inhibin A and Activin A concentrations in plasma collected from a thoroughbred mare over an estrous cycle time period;

FIG. 5 through FIG. 12 are graphs of various TGF-β superfamily peptide hormones collected from a reproductively successful Standardbred mare (mare 128) and a reproductively unsuccessful Standardbred mare (mare 231) over an eight day portion of their estrous cycles; and

FIG. 13 through FIG. 17 are graphs of inhibin A concentrations in plasma and other data collected from Arabian mares over an estrous cycle time period.

Like reference symbols in the various figures of the Drawing indicate like elements. The elements in the Drawing are not to scale.

### DETAILED DESCRIPTION

The disclosed method and system may be used for breeding purposes to better predict the time of ovulation or reproductive competence in mares. The method and system allow improved forecasting of ovulation to help reduce unsuccessful breeding attempts and the associated costs. The disclosed method and system may also be used to identify abnormal levels (viz., less than normal or greater than normal levels) of TGF-β superfamily hormones in incompetent mares and thus may be used to increase success by enabling a focus on breeding more competent mares or by facilitating the administration of supplemental (viz., contributory or offsetting) hormones to improve competence.

The disclosed method may be used with mares from a variety of equine breeds, including mares from American Saddlebred, Andalusian, Appaloosa, Arabian, Belgian, Donkey, Fresian, Gypsy Vanner, Hanoverian, Holsteiner, Lippizzaner, Miniature Horse, Morgan, Oldenburg, Paint, Percheron, Quarter Horse, Rocky Mountain, Shetland Pony, Shire, Tennessee Walker, Thoroughbred and Trakhner breeds.

A variety of TGF-β superfamily peptide hormone concentrations may be measured using the disclosed method and system. Exemplary such peptide hormones include Activins such as Activin A, Activin B and Activin AB; Anti-Müllierian hormone (AMH); bone morphogenetic proteins (BMPs) such as BMPs 2, 3, 4, 5, 6, 7, 8A, 8B, 10 and 15 (BMP2, BMP3, BMP4, BMP5, 1.31V1P6, BMP7, BMP8A, BMP8B, BMP10 and BMP15); growth differentiation factors (GDFs) such as GDFs 1, 2, 3, 3A, 5, 6, 7, 8, 9, 10 and 11 (GDF1, GDF2, GDF3, GDF3A, GDF6, GDF7, GDF8, GDF9, GDF10 and GDF11); glial cell-derived neurotrophic factors (GDNFs), Inhibins such as Inhibin A (INHA); Inhibin B (INHB); Inhibin beta A, beta B, beta C and beta B (INHBA, INHBB, INHBC and INHBE); left right determination factors 1 and 2 (LEFTY1 and LEFTY2); Modals; transforming growth factors beta 1, beta 2 or beta 3 (TGF-β1, TGF-β2 or TGF-β3); and combinations of any two or more such peptide hormones. Some TGF-β superfamily peptide hormones may be more useful for predicting the imminence of ovulation, including Activin A, Inhibin A, Inhibin. B and AMR. Some of the same as well as other TGF-β superfamily peptide hormones may be more useful for assessing competence for breeding, including AMH, BMP2, BMP5, BMP6, BMP1.5 and GDF9. Measuring the concentrations of two or more peptide hormones and examining their total concentration over time or comparing the concentration of each to itself over time may improve the accuracy of prediction of time of ovulation or competence.

Measurements may for example be performed using a variety of commercially or experimentally available techniques, with enzyme immunoassay (EIA) tests being preferred and enzyme-linked immunosorbent assay (ELISA) tests being especially preferred. EIA and ELISA test kits and instruments for testing humans or other mammals are commercially available from a number or suppliers including Abeam PLC, Ansh Labs, Beckman Coulter (for example the ACCESS™ Inhibin A Reagent Pack, cat. no. A36097, and the DTX™ 880 reader), Cell Signaling Technology, Cusabio Technology LLC, Diagnostic Systems Laboratories (for example the Inhibin A ELISA kit, cat. no. DSL-10-28100-1), DIAsource Immuno Assays SA (for example, the STRATEC GEMINI™ instrument), Dynex Technologies (for example, the DS2™ ELISA processing system), Enzo Life Sciences, Genorise Scientific Inc., Hamilton Company (for example, the ELIA NIMBUS™ assay workstation), Millipore Corp., MyBioSource Inc., Raybiotech, Inc., R&D Systems, Serotee Ltd. (for example the Ultra Sensitive Inhibin A Dimer Assay Kit, cat. no. MCA950KZZ), Sigma-Aldrich Co., Thermo Fisher Scientific (for example, the MULTISCAN™ and VARIOSCAN™ instrument lines) and USCN Life Science Inc. The protocols and procedures specified by the manufacturer for human testing may in many cases be used without significant modification for testing equine mares, including the use of appropriate standards and controls. The blood sample desirably is obtained using a minimally invasive, non-lethal sampling method. For example, approximately 1-2 ml of whole blood may be collected from the jugular vein in a mare's neck and analyzed using ELISA. Alternate blood draw sites include the transverse facial venous sinus, the cephalic vein, and the saphenous vein. For analyses based on serum or plasma separated from whole blood (for example, by using centrifuging or filtration), the whole blood draw desirably is sufficient to provide about 0.35 ml serum or plasma from each mare. The draw may be obtained by a veterinarian or by other suitably-trained personnel.

The blood, serum or plasma may be analyzed immediately, transported to a remote site such as a central laboratory or other site, or if desired be stored (for example in a refrigerator) or frozen (for example in a freezer) until later analysis, and warmed or thawed as need be when the analysis is performed. Typically however blood draws and the associated analyses will be performed successively over the course of several days. In some embodiments, the blood draws and analyses will be performed for at least three, at least four, at least five, at least six, at least seven or at least eight successive days, and for up to all or less than all of the days in a typical 19 to 26 day estrous cycle. In situations where the start or approximate start of a cycle is known (as established for example by using the disclosed tests, or by using other measures such as palpitation of the ovaries), then the frequency of sampling and analysis may be varied throughout a subsequent cycle. For example, less frequent sampling and analysis (for example, every third, fourth or fifth day, or even not at all) may be carried out near the start of a cycle, and more frequent sampling and analysis (for example, every day or every other day) may be carried out near the end of a cycle. While it may be desirable to carry out blood draws from individual mares at about the same time each day, it may be necessary to obtain blood draws from the mares in a large herd at different times during the day in order to accommodate the logistics of mare collection and care. In an embodiment, it will typically be desirable to record both the day and time each individual sample is collected, so as to take the time of day that a sample was collected into account when assessing a set of collected sample measurements.

The collected blood, serum or plasma samples may be placed in the wells of microliter plates coated with appropriate antibodies (for example, antibodies directed towards Activin A, Inhibin A, inhibin B, Mal or other members of the TGF-3 superfamily). After incubation and washing steps, a detection antibody (for example, an antibody labeled with horseradish peroxidase) may be added to the wells followed by a substrate (for example tetramethylbenzidine). Colorimetric changes may be measured at appropriate wavelengths using a microplate reader, with the measured absorbance being directly proportional to the peptide hormone concentration in the sample. Standard curves may be generated and used to determine the peptide hormone concentration in each sample. The results may be used to predict the imminence of ovulation and breeding competence generally, depending upon the chosen peptide hormone and desired mare breeding characteristic being analyzed. The above steps may be performed manually or using suitable automated instruments, with the use of automated instruments equipped with suitable capability for storage of the resulting data being preferred. Such storage may be within the instrument, in a nearby networked or otherwise connected auxiliary device, or in a cloud storage or other remote facility or service.

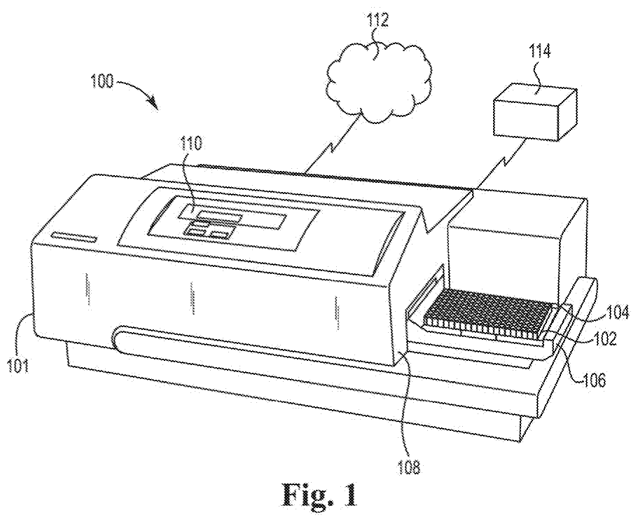

A representative system 100 for processing and measuring peptide hormone concentrations is shown in FIG. 1. Automated instrument 101 supports a microtiter plate 102 containing sample wells 104 and positioned atop movable stage 106. Once loaded with samples, stage 106 passes into housing 108 where the above-mentioned incubation, washing, antibody addition and absorbance measurement steps are performed. Control of the operation of instrument 101 can be performed and the results for selected samples can be displayed using touch panel 110. The results may be stored and processed for analysis using a suitable engine. The term engine as used herein is defined as a real-world device, component, or arrangement of components implemented using hardware, such as by an application specific integrated circuit (ASIC) or held-programmable gate array (FPGA), for example, or as a combination of hardware and software, such as by a microprocessor system and a set of program instructions that adapt the engine to implement a desired functionality, which while being executed may transform a microprocessor system into a special-purpose device. The above-mentioned measurement results may be stored within instrument 101, stored in a nearby or networked separate storage location (not shown in FIG. 1) or remotely stored using for example cloud storage facility 112. The above-mentioned engine may reside within instrument 101, within a nearby or networked separate processing device (not shown in FIG. 1) or may be remotely processed for analysis using for example remote engine and processor 114.

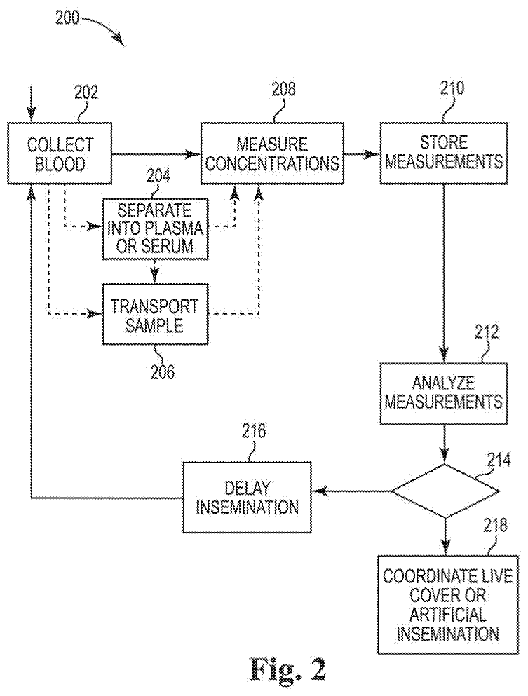

A block diagram 200 of steps that may be employed in the disclosed method is shown in FIG. 2. Whole blood is collected 202 from a mare, and optionally separated 204 to obtain plasma or serum. The blood, plasma or serum is optionally transported 206 to a measurement instrument where peptide hormone concentration measurements are obtained 208 for one or more peptide hormones. The measurements are stored 210 along with other previously or subsequently stored measurements for the same mare, using for example onboard, nearby, networked or cloud storage. The stored measurements are analyzed 212 using for example onboard, nearby, networked or cloud computing. Based on analysis 212, an alternative 214 is followed, namely to delay or forego 216 any breeding attempts this cycle, and instead continue monitoring, or to coordinate 218 live cover or artificial insemination.

The disclosed analysis may be performed using a variety of engines, each of which is constructed, programmed, configured, or otherwise adapted, to autonomously carry out a function or set of functions. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine may be realized in a variety of physically realizable configurations, and should generally not be limited to any particular implementation discussed or exemplified herein, unless such limitations are expressly called out. In addition, an engine can itself be composed of more than one sub-engine, each of which can be regarded as an engine in its own right. An engine or a variety of engines may correspond to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically discussed herein.

Various embodiments of the disclosed system, and the corresponding methods of configuring and operating the disclosed system, may be performed using cloud computing, client-server, or other networked environments, or any combination thereof. The components of the system can be located in a singular “cloud” or network, or spread among many clouds or networks. End-user knowledge of the physical location and configuration of components of the system is not required.

As will be readily understood by one of skill in the art, the disclosed system may be implemented using at least one processor and operably coupled memory. The processor can be any programmable device that accepts digital data as input, is configured to process the input according to instructions or algorithms, and provides results as outputs. In an embodiment, a processor can be a central processing unit (CPU) configured to carry out the instructions of a computer program. A processor is therefore configured to perform at least basic arithmetical, logical, and input/output operations.

Memory operably coupled to the processor can include volatile or non-volatile memory as required by the coupled processor to not only provide space to execute the instructions or algorithms, but to provide the space to store the instructions themselves. In embodiments, volatile memory can include random access memory (RAM), dynamic random-access memory (DRAM), or static random-access memory (SRAM), for example. In embodiments, non-volatile memory can include mad-only memory, flash memory, ferroelectric RAM, hard disk, floppy disk, magnetic tape, or optical disc storage, for example. The foregoing lists in no way limits the type of memory that can be used, as these embodiments are given only by way of example and are not intended to limit the scope of the disclosed system. The disclosed storage component generally includes electronic storage for data concerning the days and optionally the times at which samples have been taken and a name, number or other identifier for the mare or mares from which the samples were obtained. In an embodiment, the disclosed storage may be a general-purpose database management storage system (DBMS) or relational DBMS as implemented by, for example, Oracle, IBM DB2, Microsoft SQL Server, PostgreSQL, MySQL, SQLite, Linux, or Unix solutions, and for which SQL calls may be utilized for storage and retrieval. In another embodiment, the disclosed storage, engine or both may employ a cloud computing service such as the Amazon Web Services (AWS) cloud computing service.

The collected data is retrieved from storage and processed at a suitable time or times to enable prediction of imminent ovulation, reproductive competence or both. In one embodiment, such prediction may be based on assessment of the relative magnitude of the concentration(s) on any given day of one or more TGF-β superfamily peptide hormones, with such concentrations being measured individually or in total for such one or more hormones. By way of example, imminent ovulation may be predicted based on the observation of an at least 2, at least 3 or at least 4 picogam/ml increase from one day to the next in the measured concentration of a peptide hormone (e.g., Inhibin A) in successive whole blood, serum or plasma samples. In another embodiment, such prediction may be based on a change in the slope of successive concentration measurements over two or more days. By way of example, imminent ovulation may be predicted based on the observation of an at least 10%, at least 20%, at least 30%, at least or at least 50% change, whether positive or negative, in the relative magnitude of successive daily concentration measurements of one or more such peptide hormones in successive whole blood samples. In yet another embodiment, such prediction may be based on observation of a change from positive to negative slope or from negative to positive slope in a plot of successive daily concentration measurements of one or more such peptide hormones versus time. In a further embodiment, such prediction may be based on observation of the convergence or divergence of two overlaid plots of successive daily concentration measurements of one or more peptide hormones versus time. In yet a further embodiment, such prediction may be based on a comparison of the peptide hormone concentration for an individual mare to the concentration of such peptide hormone in another mare or to an average concentration for a group of mares, for an entire herd or for some subset thereof (e.g., a difference that is >20 pg/mL, >40 pg/mL, >45 pg/mL, >50 pg/mL, >60 pg/mL or >60 pg/mL).

For example, in one embodiment, the onset of ovulation is detected or predicted based on a change in the relative magnitude or slope of successive individual concentration measurements for a single TGF-β superfamily peptide hormone. In another embodiment the onset of ovulation is detected or predicted based on a comparison of the relative magnitude or slope of successive individual concentration measurements for one or more TGF-β superfamily peptide hormones in a single mare to the relative magnitude or slope of successive average concentration measurements for one or more TGF-β superfamily peptide hormones in a collection of mares. In a further embodiment, the onset of ovulation is detected or predicted based on a change in the relative magnitude or slope of successive individual concentration measurements for at least two TGF-β superfamily peptide hormones. In a further embodiment, reproductive competence is detected or predicted based on the magnitude of individual concentration measurements for one or more TOF-β superfamily peptide hormones compared to the average concentration measurements for a group of reproductively competent mares.

Evaluation of reproductive competence may consider a number of factors, including but not limited to suitability of an ovulation for conception and whether a particular ovulation is accompanied by necessary concurrent biological processes for conception. For example, a mare may release two eggs in a single estrus cycle, raising the chances of conceiving twins. While the successful birth of twin foals is not unheard of, it is neither common nor desirable. The gestation and delivery of twin foals poses a significant risk to both foals and to the mother. Further some instances the two eggs may be released asynchronously, or back to back rather than simultaneously (synchronous). After the first ovulation, the cervix has a tendency to Close before the second of the back to back ovulations, and more often than not causing insemination, to be unsuccessful.

Breeding via live cover or artificial insemination desirably be coordinated to occur no later than three days (and more preferably no later than two days or no later than one day) after the onset of ovulation. Such coordination may for example involve requesting, arranging or permitting mare live cover, or requesting, arranging or permitting mare artificial insemination. Such coordination may be performed by the individual responsible for collecting or analyzing the samples, or may be performed by an at least partial owner of the subject mare.

The invention is further illustrated in the following non-limiting examples, in which all parts and percentages are by weight unless otherwise indicated.

### Example 1

Whole blood samples were collected from thoroughbred or Arabian mares and simultaneous palpitations were performed during the end of their estrous cycles. The blood samples were centrifuged and plasma from each sample was analyzed using 96-well Inhibin A microplates from MyBioSource Inc., 96-well Activin A microplates from Abeam PLC and a DS2™ ELISA processing system workstation from Dynex Technologies.

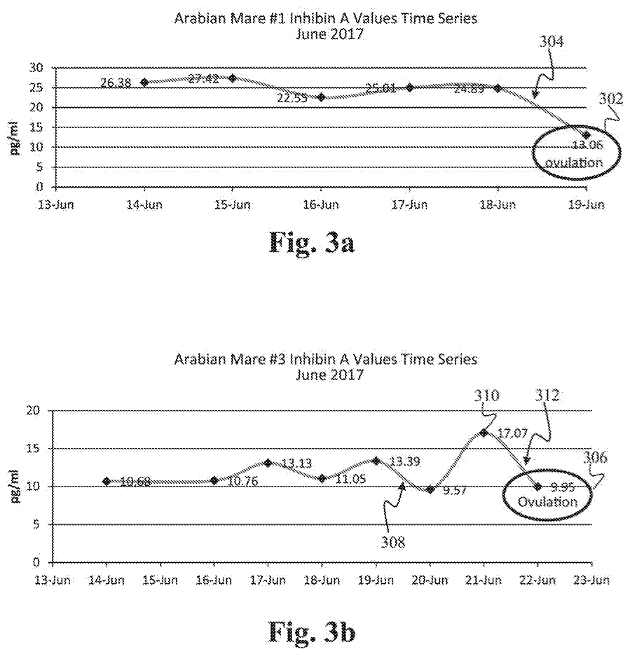

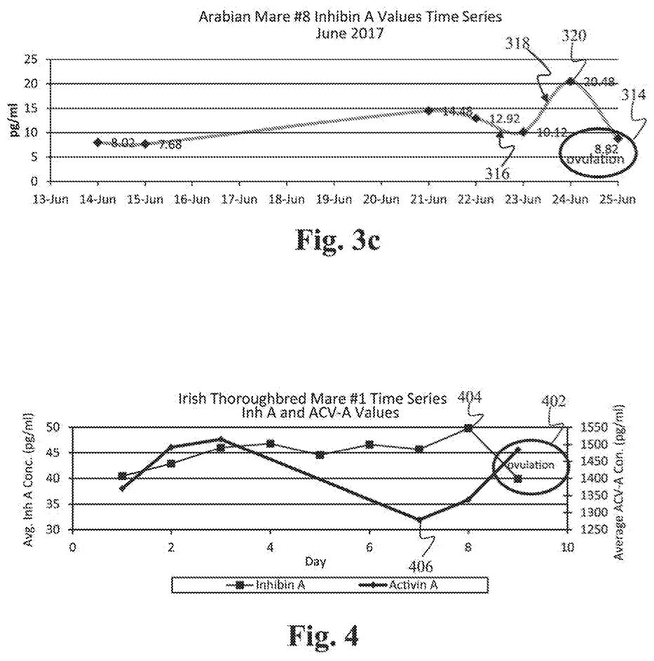

For Arabian Mare #1, inhibin A concentrations were measured daily over a 6 day period. For Arabian Mare #3, Inhibin A concentrations were measured 8 times over a 9-day period, with a 2-day gap between the first and second measurements and with daily measurements commencing with the second blood draw. For Arabian Mare #8, Inhibin A concentrations were measured 7 times over a 12 day period, with a. 5 day gap between the second and third blood draws and daily measurements thereafter. For Irish Thoroughbred #1, Inhibin A concentrations were Measured daily over a 9 day period, and Activin A concentrations were measured 6 times over the 9 day period with a 3 day gap between the third and fourth measurements and daily measurements before and after the 3 day gap. The measured and stored concentrations and dates of ovulation are shown graphically in FIG. 3a through FIG. 3c respectively for Arabian Mare #1, Arabian Mare #3 and Arabian Mare #8, and in FIG. 4 for Irish Thoroughbred #1.

The results show that significant changes in inhibin A concentration correlate to ovulation in the respective mare. For example, the results in FIG. 3a show that for Arabian Mare #1, ovulation 302 was imminent (and in fact took place) on the 6th day (June 19) as indicated by a >11 pg/mL decrease (nearly 50% of the prior day June 18 measurement) in Inhibin A concentration in plasma. The results in FIG. 3b show that for Arabian Mare #3, ovulation was imminent on the 7th day (June 20) as indicated by a >3.8 pg/mL decrease 304 (about 29% of the prior day June 19 measurement) in Inhibin A concentration in plasma. The results in FIG. 3h show that for Arabian Mare #3, ovulation 306 was imminent on the 7th day (June 20) as indicated by a >3.8 pg/mL decrease 308 (about 29% of the prior day June 19 measurement) in Inhibin A concentration in plasma. This was followed by a >7.5 pg/mL spike MO (about 78% of the prior day June 20 measurement) and a >7.1 pg/mL decrease 312 (about 42% of the prior day June 21 measurement) up through the June 23 ovulation date.

The results in FIG. 3c show that for Arabian Mare #8, ovulation 314 was imminent on the 10th day (June 23) as indicated by a >2.8 pg/mL decrease 316 (about 22% of the prior day June 22 measurement) in Inhibin A concentration in plasma. This was followed by a >10.4 pg/mL increase 318 (about 102% of the prior day June 23 measurement) and an 11.7 pg/mL decrease (about 57% of the prior day June 24 measurement) up through the June 25 ovulation date. Like the data shown in FIG. 3b, the FIG. 3c results show a distinct “spike” 320 in Inhibin A concentration just prior to ovulation.

The results in FIG. 4 show that for Irish Thoroughbred #1, ovulation. 402 was imminent on the 8th day as indicated by a reversal in slope 406 (from negative over the prior four day endpoint-sampled period to positive on the fifth day) in the Activin A concentration in plasma. The Inhibin A and Activin A concentration curves were also observed to converge at day 8 and then cross one another at day 9, which also marked the onset of ovulation 402. On the 9th day, a 10 pg/mL decrease in Inhibin A concentration and a 145 pg/mL increase in Activin A concentrations were also observed. The characteristic spike 404 in Inhibin A is visible on day 8 just prior to ovulation 402.

### Example 2

In preparation for artificial insemination, whole blood samples were collected from two American Standardbred Horses (mare #128 and mare #231) during the last week of their estrous cycles. Blood samples were obtained 7 times over an 8 day period, with a. 2 day gap between the first and second blood draws and with daily blood draws thereafter. Simultaneous observations by palpitation indicated that ovulation 502, 504 occurred in both mares on June 2.

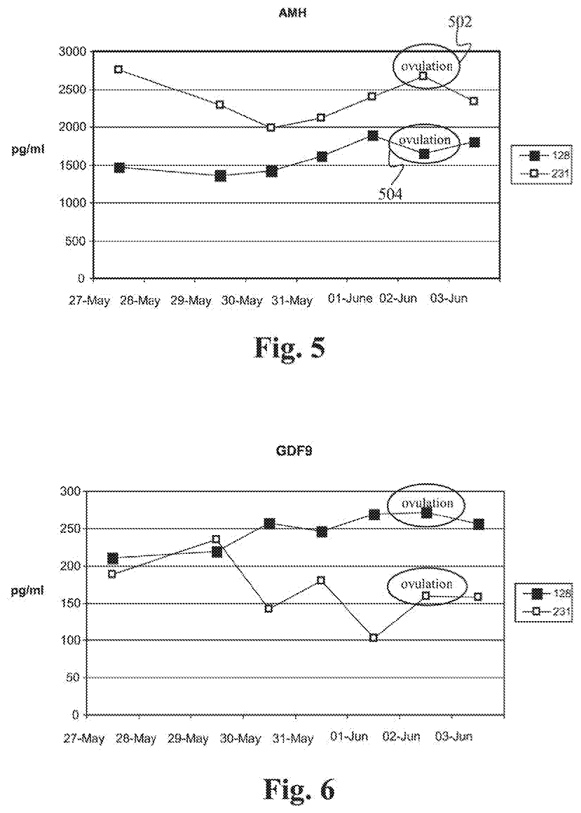

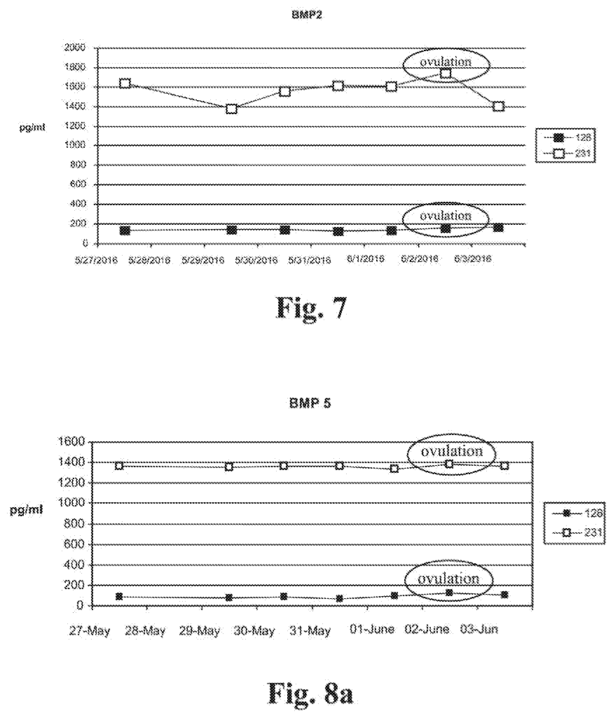

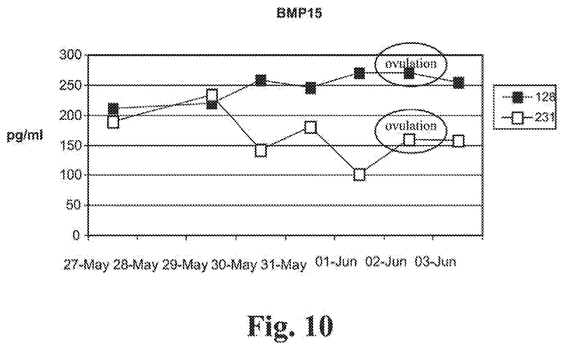

The blood samples were centrifuged and plasma from each sample was analyzed. The analyses were performed using 96-well microplates from several sources identified below and the DS2 workstation employed in Example 1. This enabled determination and storage of successive concentrations in each mare for several members of the TGF-β superfamily of peptide hormones. The stored results are shown in FIG. 5 (Ansh Labs AMH microplates), FIG. 6 (MyBioSource Inc. GDF9 microplates), in FIG. 7 through FIG. 9 (BMP2, BMP5 and BMP6 microplates from Genorise Scientific Inc.), in FIG. 10 (MyBioSource Inc. BMP15 microplates).

Mare #128 successfully produced a live foal and thus appeared to be reproductively competent. Mare 231 did not conceive. The results in FIG. 5 may be useful for detecting the imminence of ovulation, as there was a consistent increase in AMH concentrations for both mares beginning around three days prior to ovulation 502, 504. Some but perhaps not all of the results in FIG. 6 though FIG. 10 may be useful for assessing reproductive competence, based on the appreciable divergence in the magnitude or direction of change in measured concentrations for some of the measured peptide hormones in mares #128 and #231.

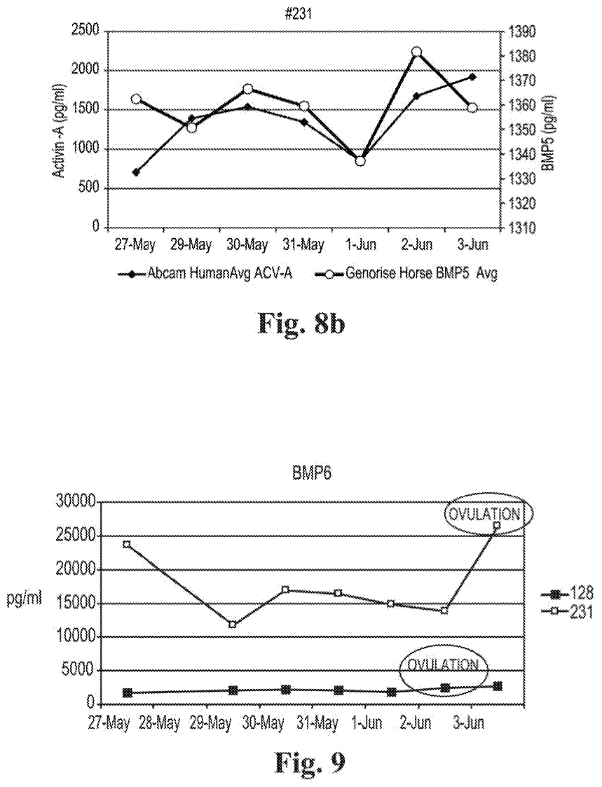

A noteworthy correlation between Activin A and BMP5 was observed in Mare #231, depicted in FIG. 8b, and indicating that BMP5 may be another useful biomarker.

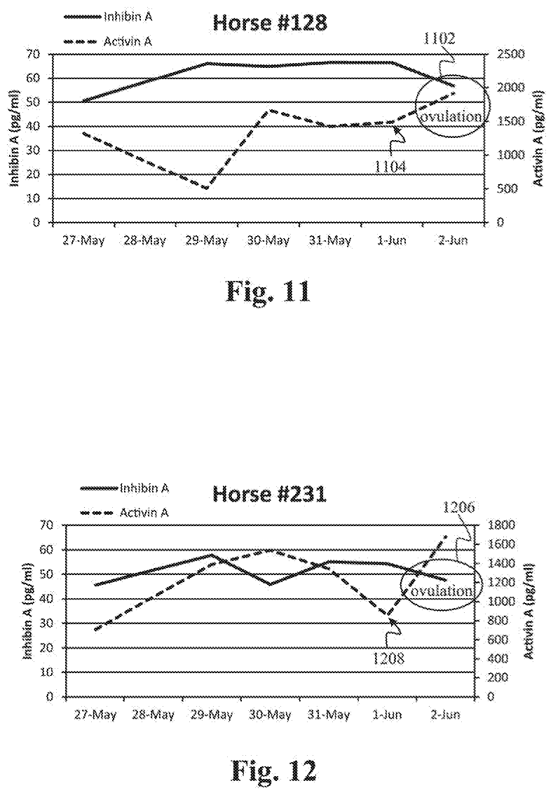

As shown in FIGS. 11 and 12, inhibin A and Activin A measurements for mares #128 and #231 exhibit convergence at ovulation 1102, 1206 or crossover behavior like that shown in FIG. 4, as well as pre-ovulation slope changes 1104, 1208 in Activin A concentrations.

### Example 3

Blood samples were taken twice a day from ten Arabian mares over a 40 day period. The samples were evaluated using the Inhibin A and Activin A microplates and the DS2 workstation employed in Example 1. Progesterone concentration analysis was also performed using an ELISA progesterone assay from Cayman Chemical. For comparison, periodic palpation and edema measurements made by a veterinarian provided traditional tracking of ovulation and identification of a target coordination window for breeding.

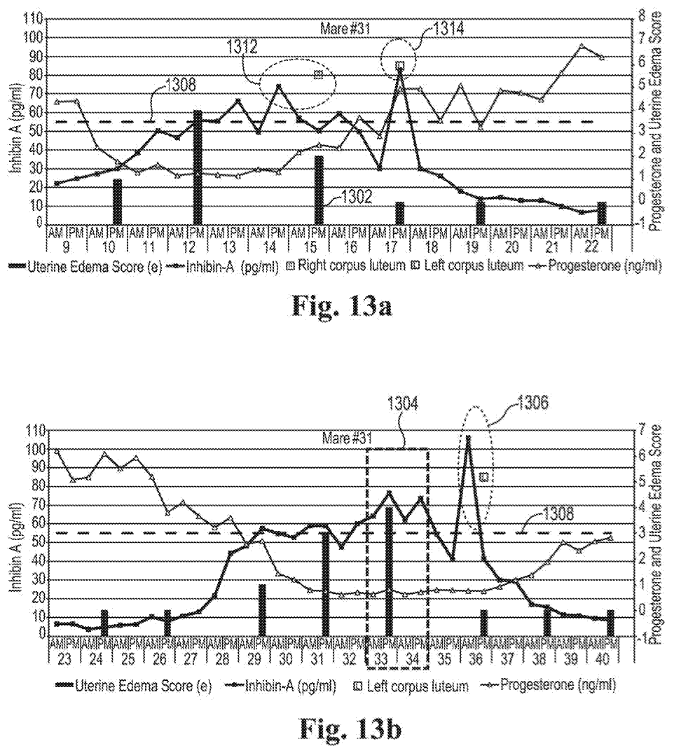

Mare #31 exhibited two ovulations over the course of the 40 days. The first ovulation of the 40-day sampling period, days 9-22, depicted in FIG. 13a, shows the response of Inhibin A in the case of an asynchronous dual ovulation. The second ovulation of the 40-day period, days 23-40, depicted in FIG. 13b, shows the response of Inhibin A in the case of a single ovulation.

Both FIGS. 13a and 13b depict inhibin A (black squares, units of pg/mL, left axis) and progesterone (white triangles, units of ng/mL, right axis) as line graphs. Uterine edema is depicted as a grey bar graph 1302, ranging from 0 to a max score of 4 (right axis), Left and right ovary corpus luteum are denoted with L and R symbols corresponding to when corpus luteum were first documented to be present on either ovary during a cycle. A dashed line box 1304 in FIG. 13b denotes the approximately two-day window identified by the veterinarian, according to palpation measurement and edema score, as a desirable target coordination window. A dashed line 1308 identifies an Inhibin threshold that may be used to identify when a mare will ovulate imminently or when the target coordination window is open. In this example, the threshold 1308 is set at Inhibin=55 pg/mL, but could be adjusted to be more conservative (lower value) or more liberal (higher value) depending on the needs of the vet or breeder, Other embodiments may set different values for the threshold 1308, according to the particular goals of the user or the particular biomarker characteristics of the subject mare.

The coordination window can be identified by a veterinarian due to increased edema, in response to rising estrogen, and increased size in the maturing follicle(s). Ovulation can be confirmed to have occurred at some point, by evidence of reduced edema and detection of the corpus luteum by palpation. Meanwhile, referring to FIG. 13b, Inhibin A shows a marked increase in the days before ovulation, rising together with the edema. First discernible from the evening of day 27 to the morning of day 28, by day 29 Inhibin A is clearly elevated, a full 4 days prior to the target coordination window 1304. Inhibin A can also be seen to drop precipitously, along with edema, following a spike 1306 on day 36 following ovulation (ovulation from left ovary likely occurred sometime from days 33 to 35, confirmed by detection of corpus luteum by palpation at day 36). Thus, Inhibin A concentration changes appear to track well with physiological indicators used by veterinarians to try to help target a breeding coordination window, generally 2.4-48 hours before ovulation. Inhibin A analyses are able to provide a much earlier prediction for ovulation than the physiological changes currently used by veterinarians.

The above data may also be compared to Mare #31's first ovulation in the 40 day observation period, shown in FIG. 13a. Here there is an observable rise to a peak 1312 followed by ovulation from the right ovary (days 9-15). However, instead of a sharp drop from peak 1312 and a continued decline, as seen in FIG. 13b at 1306 with a single ovulation, the decline is attenuated and rises to a second peak 1314 at day 17 as the left ovary also ovulates (days 15-17), This demonstrates changes in the readings of Inhibin A when an atypical asynchronous dual ovulations has occurred which can result in unsuccessful fertilization. Further asynchronous ovulation patterns of Inhibin A concentrations appear to be distinct from the synchronous ovulation patterns of Inhibin A concentrations, which is significant given the different concerns associated with each dual ovulation type. Thus, the disclosed method may reveal not only when a target breeding coordination window opens or persists, but may also provide a qualitative indication of the potential reproductive outcome associated with a particular ovulation.

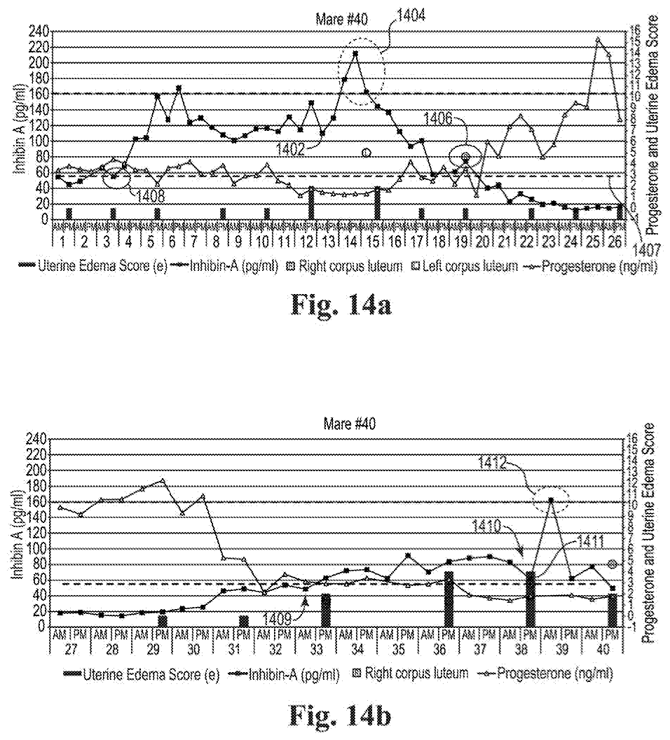

For example, turning now to FIG. 14a, depicted is data from another asynchronous dual ovulation for Mare #40 from the above-described study. A first ovulation likely occurred around days 12-13, associated with a noticeable dip 1402 in otherwise rising Inhibin A and confirmed by detection of a left corpus luteum at day 15 directly before the identified sharp peak 1404 in Inhibin A, rising to about 215 pg/mL for this mare. Formation of a second corpus luteum (in the right ovary) was confirmed on day 19 with an associated second spike 1406 in Inhibin A of about 75 pg/mL. As shown for the depicted dual asynchronous ovulation, Inhibin A begins rising as much as 11 days prior to ovulation, crossing the example threshold 1407 at day 3 1408, and reaches significantly higher values than a single ovulation in the same mare, shown in FIG. 14b.

In FIG. 14b, a single ovulation in Mare #40, Inhibin A begins to rise 1409 only about 8 days before ovulation 1410 (day 38, according to max edema 1411 on day 38 and the spike 1412 in Inhibin A on day 39), and does so at a significantly more moderate rate than in the dual asynchronous ovulation of FIG. 14a. The single ovulation peak for Inhibin A 1412 is much lower as well, only reaching about 160 pg/mL. The higher values and faster accumulation seen in FIG. 14a appear to correlate well with dual asynchronous ovulations, permitting early identification of a potentially unsuccessful insemination.

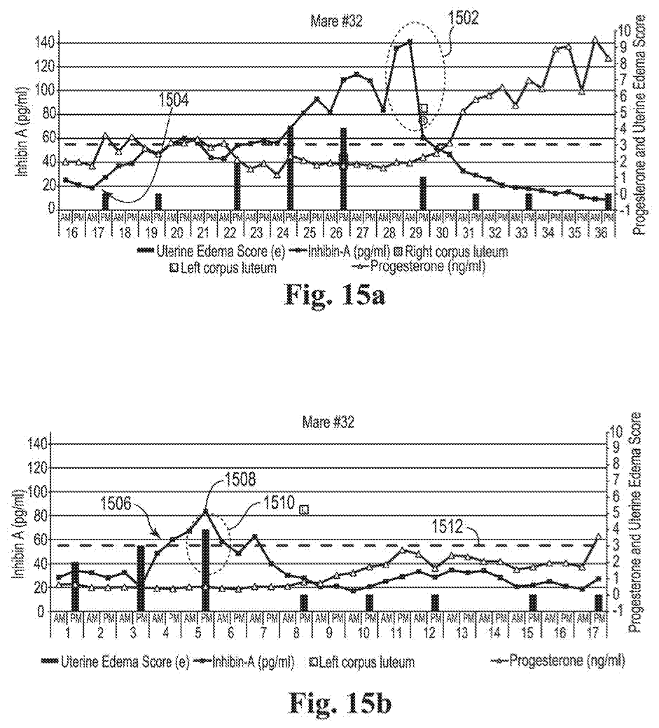

Referring now to FIG. 15a, Mare 432 experienced a synchronous dual ovulation. 1.502 around day 28, but Inhibin A began rising 1504 as early as day 17, nearly 11 days prior. In the case of the dual synchronous ovulation 1502, Inhibin A peaked at 140 pg/mL. Compared with the slope 1506 and peak 1508 for a single ovulation 1510 in the same mare (Mare #32), seen in FIG. 15b, the difference is telling. In the case of a single ovulation 1510 in Mare #32, Inhibin A peaked 1508 at a little over 80 pg/mL, barely over half the peak 1502 for a dual synchronous ovulation, and began to rise 1506 only about 4 days before ovulation occurred. In FIG. 15b, the threshold 1512 is set at about 55 ng/mL of Inhibin A, and represents an embodiment in which the threshold is set to indicate when the target breeding coordination window opens.

As mentioned above, breeding a mare during an estrus with dual ovulations significantly raises the odds of twin foals. Twin pregnancies in mares place all three horses at considerable risk, and good outcomes are rare. Further, in the case of an asynchronous double ovulation, the two fetuses may be different ages and thus different sizes. It may be difficult to identify the second, smaller, fetus early in the pregnancy, resulting in late identification of the twins and limiting the options for veterinary intervention. Early identification of a double ovulation raises the chances of avoiding a twin pregnancy or facilitates early veterinary intervention before the mare or either foal is in danger.

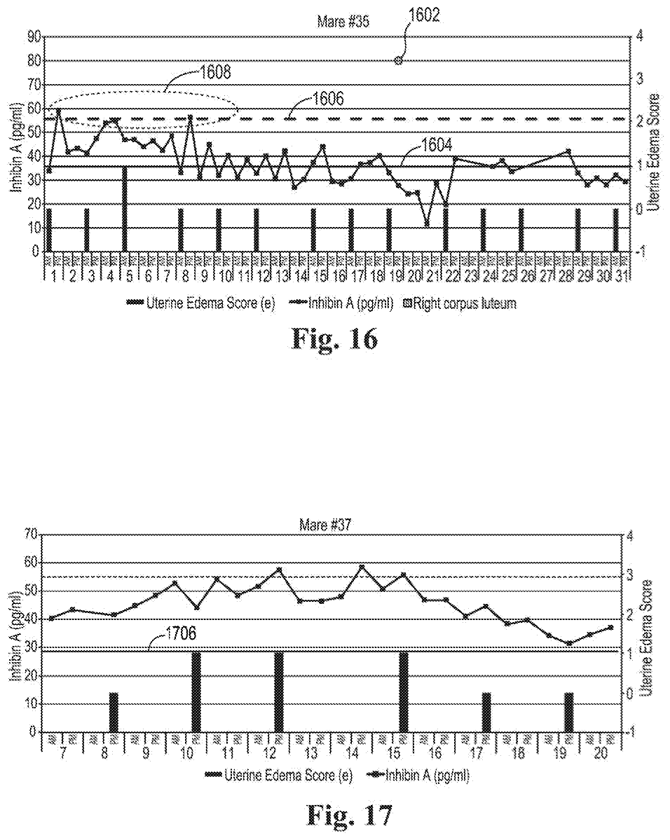

FIGS. 16 and 17 demonstrate the behavior of Inhibin A in mares with low reproductive competence for a particular cycle, and which may be unlikely to conceive. Looking first at FIG. 16, Mare 435 did produce a corpus luteum from the right ovary around day 17 1602. However, no uterine edema score rose higher than 1 (see threshold 1604), whereas a score of 4 is often necessary for confirmation that the uterus is properly preparing to carry a foal. The lack of any significant uterine edema indicates Mare #35 is unlikely to conceive this cycle, even if an egg was released as suggested by the corpus luteum 1602. This low reproductive competence is also reflected in the measurements for Inhibin A, which at no point show the extended rise and significant spike associated with ovulation in Mares #31 (see FIG. 13b at 1306), #40 (see FIG. 14b at 1409 and 1412), and #32 (see FIG. 15b at 1506 and 1508). Instead, the Inhibin A level in Mare #35 remains relatively flat, only barely rising 1608 above example threshold 1606 of 55 pg/mL, and failing to sustain the rise as in Mares #31, #40, and #32. Likewise, Mare #37's data for Inhibin A, shown in FIG. 17, shows neither a sustained rise nor a distinct spike. The low values measured for uterine edema, not exceeding a score of one 1706, confirms the low reproductive competence of this mare suggested by the behavior of Inhibin A.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1-70. (canceled)

71. A method for assessing the imminence of mare ovulation and reproductive competence comprising:
collecting samples of blood, serum, or plasma from a mare over at least two days in the mare's estrous cycle;
obtaining a set of concentration measurements of one or more peptide hormones of the transforming growth factor-beta superfamily (TGF-β superfamily) in the samples over the at least two days;
detecting an onset of a change in a relative magnitude or slope of successive concentration measurements of the one or more peptide hormones during the at least two days; and
coordinating live cover or artificial insemination of the mare to occur no later than three days after the onset of the change.

72. A method according to claim 71, wherein the peptide hormone comprises Inhibin A.

73. A method according to claim 71, wherein the peptide hormone comprises Activin A.

74. A method according to claim 71, wherein the peptide hormone comprises Inhibin B or Activin B.

75. A method according to claim 71, wherein the peptide hormone comprises one or more Anti-Müllerian hormone (AMH), bone morphogenetic protein (BMP) or growth differentiation factor (GDF).

76. A method according to claim 71, wherein the onset is detected based on a comparison of the relative magnitude or slope of successive individual concentration measurements for one or more TGF-β superfamily peptide hormones in a single mare to the relative magnitude or slope of successive average concentration measurements for one or more TGF-β superfamily peptide hormones in a collection of mares.

77. A method according to claim 71, wherein the onset is detected based on an at least 20% change in the relative magnitude of successive daily concentration measurements of one or more such peptide hormones.

78. A system for assessing the imminence of mare ovulation and reproductive competence, comprising:
a detector configured to measure concentrations of one or more peptide hormones of the TGF-β superfamily in samples of blood, serum, or plasma obtained over at least two days during a mare's estrous cycle;
a processor;
a memory for containing a set of measured concentrations and sampling dates of the peptide hormones in the samples; and
an engine for detecting an onset of a change in a relative magnitude or slope of successive concentration measurements of the one or more peptide hormones over the two or more days.

79. The system according to claim 78, wherein detecting an onset of change in the relative magnitude or slope of successive concentration measurements indicates a likelihood that the mare will exhibit low reproductive competence during the present estrous cycle.

80. A system according to claim 78, wherein the detector is configured to measure concentrations of Inhibin A.

81. A system according to claim 78, wherein the detector is configured to measure concentrations of one or more of Inhibin B, Activin A, and Activin B.

82. A system according to claim 78, wherein the onset is detected based on a change in the relative magnitude or slope of successive individual concentration measurements for a single TGF-β superfamily peptide hormone.

83. A system according to claim 78, wherein the onset is detected based on a change in the relative magnitude or slope of successive measurements of the combined total concentration of at least two TGF-β superfamily peptide hormones.

84. A system according to claim 78, wherein the onset is detected based on a crossover of the concentration measurements of at least two TGF-β superfamily peptide hormones.

85. A system according to claim 78, wherein the set comprises concentration measurements obtained at least daily over two or more days of the mare's estrous cycle, for at least Inhibin and Activin peptide hormones.

86. A system according to claim 85, wherein the set comprises concentration measurements obtained at least daily over two or more days of such estrous cycle, for at least Inhibin A and Activin A peptide hormones.

87. A method for assessing the imminence of mare ovulation and reproductive competence, which method comprises:
collecting blood, serum or plasma samples from a mare over two or more days in such mare's estrous cycle;
obtaining a set of concentration measurements of one or more peptide hormones of the transforming growth factor-beta superfamily (TGF-β superfamily) in such samples over such two or more days;
calculating a change in the set of concentration measurements over such two or more days;
determining that the change in the set of concentration measurements over such two or more days exceeds a threshold; and
coordinating live cover or artificial insemination of such mare to occur in response to the change in the set of concentration measurements over such two or more days exceeding the threshold.

88. The method according to claim 87, wherein the set of concentration measurements includes concentrations of one or both of Inhibin A and Activin A.

89. The method according to claim 87, wherein the threshold is a concentration measurement of >55 pg/mL.

90. The method according to claim 87, wherein the threshold is a concentration measurement of >160 pg/mL.
